(19) United States
(12) Patent Application Publication
(10) Pub. No.: US 2023/0149339 A1
(43) Pub. Date: May 18, 2023

(54) Compositions And Methods For Treating Feeding Tube Patients

(71) Applicant: MGS Products LLC, Hellertown, PA (US)

(72) Inventor: Tracy E. Hill, Hellertown, PA (US)

(21) Appl. No.: 18/055,936

(22) Filed: Nov. 16, 2022

Related U.S. Application Data
(60) Provisional application No. 63/279,725, filed on Nov. 16, 2021.

(51) Int. Cl. A61K 31/25; A61K 45/06; A61K 9/00

(52) U.S. Cl. CPC A61K 31/25; A61K 45/06; A61K 9/006

(57) ABSTRACT

The disclosure provides compositions for moistening an oral mucous membrane of a patient in need thereof, comprising propylene glycol and a flavoring agent. Devices comprising the compositions described herein also are provided. The disclosure also provides methods for moistening an oral mucous membrane of a patient in need thereof, methods for stimulating the appetite of a patient in need thereof, and methods for improving a disposition of a patient unable to swallow food or excess liquid using the compositions or devices described herein.

### CROSS-REFERENCE TO RELATED APPLICATIONS

This application claims priority to U.S. Provisional Patent Application No. 63/279,725, filed Nov. 16, 2021, the entire disclosure of which is incorporated by reference herein.

### TECHNICAL FIELD

The disclosure relates to compositions and methods for treating feeding tube patients.

### BACKGROUND

There are more than 500,000 patients in the United States who require feeding tubes. Additionally, there are more than 50 million individuals intubated each year globally. Of the half a million patients, about 40% are pediatric patients, with 200,000 new pediatric cases per year.

Feeding tubes may be in place for a variety of medical reasons. Some patients have difficulties swallowing, an altered level of consciousness, eating disorder, or other issues that make eating challenging. For example, certain patients are unable to swallow without choking. Also, patients on total parenteral nutrition (TPN), peripheral parenteral nutrition (PPN) or enteric nutrition (ENT) have their nutritional intake administered either intravenously or gastrointestinally, through a G tube or Peg Tube. As such, said patients are not actually chewing, tasting or smelling food.

Although feeding tubes provide the necessary nutrition needed for survival, there are several downsides. Unfortunately, the feeding tube often can be a permanent solution to underlying issues, with some patients requiring feeding tubes for years, if not decades. In many cases, feeding tube patients have a decline in emotional and overall wellbeing as a result of not associating taste and smell with appetite satiation. Further, a certain amount of food or water, including saliva, may initiate gagging or choking in certain patients. However, all patients with feeding tubes eventually develop overly dry oral cavities, thereby necessitating manual wetting using devices such as swabs or dissolvable and consumable films. Further, many patients become depressed or have a poor disposition due to the failure to be able to eat, drink, taste or smell their food often correlated with emotional wellbeing from nutritional satiation. The emotional distress associated with positive nutritional satiation has been correlated with increased hospital stays, increased periods of tube feedings and increased time for healing. However, it is imperative that such patients cannot eat or drink, regardless of their cognitive state and some patients thus continue on an emotional decline.

Accordingly, there is a need in the art to manage the use of feeding tubes in patients who cannot swallow and are emotionally affected by not eating or drinking.

### SUMMARY

In some aspects, the disclosure provides compositions for moistening an oral mucous membrane of a patient in need thereof, comprising propylene glycol and a flavoring agent.

In other aspects, the disclosure provides devices comprising the composition described herein.

In further aspects, the disclosure provides methods for moistening an oral mucous membrane of a patient in need thereof, comprising applying the composition or device described herein to the oral mucous membrane of the patient.

In yet other aspects, the disclosure provides methods for stimulating the appetite of a patient in need thereof, comprising applying the composition or device described herein to the oral mucous membrane of the patient.

In still further aspects, the disclosure provides methods for improving a disposition of a patient unable to taste, smell, chew and/or swallow food or excess liquid, comprising applying the composition or device described herein to the oral mucous membrane of the patient.

Other aspects and embodiments of the invention will be readily apparent from the following detailed description of the invention.

### DETAILED DESCRIPTION OF ILLUSTRATIVE EMBODIMENTS

The compositions described herein are useful in mitigating the physical and psychological effects of tube feedings in patients. The physical advantages of the compositions include moisturizing the patient's dry mouth, which is a common problem for patients having feeding tubes. The compositions also provide taste and smell for the patient, thereby resulting in arousing the sense of taste and smell for the patient. Doing so increases the psychological and emotional wellbeing of the patient. Thus, the compositions dramatically improve the patient's life, enhance their nutritional experience and decrease their recovery or hospital time. Moreover, those patients who continue their tube feedings at home can utilize the compositions to make their home feeding times a more pleasurable experience emotionally and physically by using compositions, swabs, and films described herein.

In the disclosure, the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “approximately” it will be understood that the particular value forms another embodiment. In general, use of the term “about” or “approximately” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “approximately.” In other cases, the gradations used in a series of values may be used to determine the intended range available to the term “about” or “approximately” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude an optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself

The terms “subject” and “patient” are used interchangeably and include, without limitation, humans. In some embodiments, the patient or subject is an adult, i.e., 18 to 64 years of age. In other embodiments, the patient or subject is a geriatric adult, i.e., 65 years of age or older. In further embodiments, the patient or subject is child, i.e., a pediatric patient, 17 years of age or younger.

Compositions

The compositions are for moistening an oral mucous membrane of a patient in need thereof. The term “moistening” as used herein refers to wetting the oral mucous membrane, e.g., a dry mouth. The terms “oral mucous membrane” and “oral mucosa” are used interchangeably herein and refer to the lining mucosa, the alveolar mucosa, masticatory mucosa, and specialized mucosa. In some embodiments, the oral mucous membrane is the lining mucosa such as nonkeratinized stratified squamous epithelium, found almost everywhere in the oral cavity, including the alveolar mucosa, buccal mucosa, and labial mucosa. In certain aspects, the oral mucous membrane is the alveolar mucosa, e.g., the lining between the buccal and labial mucosae. In other aspects, the mucous membrane is the buccal mucosa, e.g., the inside lining of the cheeks and floor of the mouth. In further aspects, the mucous membrane is the labial mucosa, e.g., the inside lining of the lips. In other embodiments, the mucous membrane is the masticatory mucosa such as keratinized stratified squamous epithelium, found on the dorsum of the tongue, hard palate, and attached gingiva. In further embodiments, the mucous membrane is the specialized mucosa such as in the regions of the taste buds on lingual papillae on the dorsal surface of the tongue.

Desirably, the mucous membranes moistened using the compositions described herein contain salivary glands. Examples of salivary glands include, without limitation, parotid glands, submandibular glands, sublingual glands, tubarial glands, minor salivary glands, or von eber's glands. Because many of the treated patients are unable to swallow, the compositions provide the necessary moistening that tube fed patients need for a healthy oral cavity and teeth.

Desirably, the compositions described herein lack glycerin. As used herein, the terms “glycerin” and “glycerol” are used interchangeably and refer to propane-1,2,3-triol. In some embodiments, the compositions contain less than about 5% v/v of glycerin. In other embodiments, the compositions contain less than about 4%, about 3%, about 2%, about 1%, about 0.05%, or about 0.01% v/v of glycerin. In further embodiments, the compositions contain 0% v/v of glycerin.

The compositions contain propylene glycol. Propylene glycol is desirable for use in the instant compositions since it lacks a taste, flavor and odor and, thus, does not interfere with the taste or smell of the flavoring agent. In particular, propylene glycol lacks a taste or flavor as compared to glycerol. Additionally, glycerol is often prepared with soybean oil. Because some people have allergies to soy (which is considered a legume and part of the nut allergy family), oral ingestion of products containing glycerol can have repercussions, some of which are physical discomfort and/or can be dangerous. Propylene glycol, on the other hand, is a synthetically manufactured water soluble substance containing no known food allergens. Further, propylene glycol is quite inexpensive, thereby making it a desirable component in the instant compositions. Thus, the use of propylene glycol over glycerin will significantly cut expenses for hospitals, nursing homes, pediatric centers and the like.

The amount of propylene glycol is an amount that is sufficient to moisten the oral mucous membrane of a patient. In some embodiments, the compositions contain about 5% v/v, based on the volume of the composition, or greater of propylene glycol. In other embodiments, the compositions contain about 5, about 10, about 15, about 20, about 25, about 30, about 35, about 40, about 45, about 50, about 55, about 60, about 65, about 70, about 75, about 80, about 85, about 90, about 95, or about 99% v/v, based on the volume of the composition, of propylene glycol. In further embodiments, the compositions contain about 5 to about 99, about 5 to about 90, about 5 to about 85, about 5 to about 80, about 5 to about 75, about 5 to about 70, about 5 to about 65, about 5 to about 65, about 5 to about 60, about 5 to about 55, about 5 to about 50, about 5 to about 45, about 5 to about 40, about 5 to about 35, about 5 to about 30, about 5 to about 25, about 5 to about 20, about 5 to about 15, about 5 to about 10, about 10 to about 99, about 10 to about 90, about 10 to about 85, about 10 to about 80, about 10 to about 75, about 10 to about 70, about 10 to about 65, about 10 to about 65, about 10 to about 60, about 10 to about 55, about 10 to about 50, about 10 to about 45, about 10 to about 40, about 10 to about 35, about 10 to about 30, about 10 to about 25, about 10 to about 20, about 10 to about 15, about 20 to about 99, about 20 to about 90, about 20 to about 85, about 20 to about 80, about 20 to about 75, about 20 to about 70, about 20 to about 65, about 20 to about 65, about 20 to about 60, about 20 to about 55, about 20 to about 50, about 20 to about 45, about 20 to about 40, about 20 to about 35, about 20 to about 30, about 20 to about 25, about 30 to about 99, about 30 to about 90, about 30 to about 85, about 30 to about 80, about 30 to about 75, about 30 to about 70, about 30 to about 65, about 30 to about 65, about 30 to about 60, about 30 to about 55, about 30 to about 50, about 30 to about 45, about 30 to about 40, about 30 to about 35, about 40 to about 99, about 40 to about 90, about 40 to about 85, about 40 to about 80, about 40 to about 75, about 40 to about 70, about 40 to about 65, about 40 to about 65, about 40 to about 60, about 40 to about 55, about 40 to about 50, about 40 to about 45, about 50 to about 99, about 50 to about 90, about 50 to about 85, about 50 to about 80, about 50 to about 75, about 50 to about 70, about 50 to about 65, about 50 to about 65, about 50 to about 60, about 50 to about 55, about 60 to about 99, about 60 to about 90, about 60 to about 85, about 60 to about 80, about 60 to about 75, about 60 to about 70, about 60 to about 65, about 70 to about 99, about 70 to about 90, about 70 to about 85, about 70 to about 80, about 70 to about 75, about 80 to about 99, about 80 to about 90, about 80 to about 85, about 90 to about 99, or about 90 to about 95% v/v, based on the volume of the composition, of propylene glycol. In yet other embodiments, the compositions contain about 7.5% v/v, based on the volume of the composition, of propylene glycol.

The compositions also contain a flavoring agent. The term “flavoring agent” as used herein refers to chemical compounds that impart a specific flavored taste when administered to a patient. In some embodiments, the flavoring agent is a natural flavoring agent, nature-identical flavoring substance, or artificial flavoring agent. Examples of natural flavoring agents include substances that are obtained from plant or animal raw materials. Examples of nature-identical flavoring agents include substances obtained by synthesis or isolated through chemical processes, which are chemically and organoleptically identical to flavoring substances naturally present in products intended for human consumption. Examples of artificial substances include substances not identified in a natural product intended for human consumption, whether or not the product is processed. The flavoring agent may comprise a single ingredient/chemical compounds or may be a mixture of two or more compounds. In certain aspects, the flavoring agent contains manzanate (apple); diacetyl, acetylpropionyl, acetoin (butter); isoamyl acetate (banana); benzaldehyde (bitter almond, cherry); cinnamaldehyde (cinnamon); ethyl propionate (fruit); methyl anthranilate (grape); limonene (orange); ethyl decadienoate (pear); allyl hexanoate (pineapple); ethyl maltol (sugar, cotton candy); 2,4-dithiapentane (truffle); ethylvanillin (vanilla); or methyl salicylate (wintergreen); among others. See, e.g., the flavoring agents set forth in Helmenstine, “Food Additives List—Names and Uses,” NutritionData.com, May 19, 2021, the disclosure of which is incorporated by reference.

The flavoring agent may be selected by the patient based on the particular flavor that the patient desired to taste. For example, the patient may select the flavor from selections available based on the particular flavor the patient desired to taste, i.e., a menu or brochure. The flavoring agent selected may be based on the particular flavor that would complement the tube feeding. In certain aspects, the flavoring agent may be selected depending on the time of day or night. In other aspects, if the tube feeding is breakfast, the swab or consumable film may be breakfast-flavored. In further aspects, if the tube feeding is lunch, the swab or consumable film may be lunch-flavored. In yet other aspects, if the tube feeding is dinner, the swab or consumable film may be dinner-flavored. In still further aspects, if the tube feeding is a snack, the swab or consumable film may be snack-flavored.

In some embodiments, the flavoring agent has a sweet, sour, bitter, or spicy flavor/taste, among others. In other embodiments, the flavoring agent imparts the flavor/taste of one or more of a fruit, vegetable, beverage, meat, spice, extract, dairy product, starch, among others. In further embodiments, the flavoring agent has the flavor of a specific food substance, such as coffee, pizza (e.g., cheese pizza or pepperoni pizza), taco, or vanilla ice cream, beer, wine, burrito (e.g., breakfast burrito), cake (e.g., chocolate cake), hamburger, French fries, hamburger/French fries, cookies (e.g., chocolate chip cookies), pancakes, bacon, pancakes/bacon, macaroni and cheese or the like. The flavoring agent may differ from patient to patient and, thus, can be tailored to the patient's preferences. In other embodiments, the patient would be able to select a flavor from a list of flavors that are of most interest to the general public as determined by e.g., research studies, surveys, among others. In other embodiments, the flavor is similar to food provided by hospitals. In certain aspects, the flavoring agent is not a lemon flavor. In other aspects, the flavoring agent is not a mint flavor.

The flavoring agent optionally has an odor/scent/smell, such as an odor that matches the flavor composition. In certain aspects, the smell is directly linked to the flavor of the flavoring agent. In other aspects the flavor is linked to the smell of the flavoring agent. For example, the flavoring agent would have the same smell as the taste/flavor. By doing so, the patient is able to taste and smell the desired food, etc. By way of example, if the flavoring agent tasted like an orange, it may also smell like an orange.

Desirably, the amount of flavoring agent is selected to elicit oral moisturization without creating an abundance of saliva. The flavoring agent also stimulates the patient's sense of taste and smell, depending on the flavoring agent selected. Because the amount of flavoring agent is important in patients lacking the ability to swallow, the compositions have enough flavoring agent to elicit oral moisturization, but not too much to elicit over secretion of salivation.

The compositions contain at least about 5% v/v, based on the volume of the composition, of the flavoring agent. In some embodiments, the compositions contain about 5, about 10, about 15, about 20, about 25, about 30, about 35, about 40, about 45, about 50, about 55, about 60, about 65, about 70, about 75, about 80, about 85, about 90, about 95, or about 99% v/v, based on the volume of the composition, of the flavoring agent. In other embodiments, the compositions contain about 5 to about 99, about 5 to about 90, about 5 to about 85, about 5 to about 80, about 5 to about 75, about 5 to about 70, about 5 to about 65, about 5 to about 65, about 5 to about 60, about 5 to about 55, about 5 to about 50, about 5 to about 45, about 5 to about 40, about 5 to about 35, about 5 to about 30, about 5 to about 25, about 5 to about 20, about 5 to about 15, about 5 to about 10, about 10 to about 99, about 10 to about 90, about 10 to about 85, about 10 to about 80, about 10 to about 75, about 10 to about 70, about 10 to about 65, about 10 to about 65, about 10 to about 60, about 10 to about 55, about 10 to about 50, about 10 to about 45, about 10 to about 40, about 10 to about 35, about 10 to about 30, about 10 to about 25, about 10 to about 20, about 10 to about 15, about 20 to about 99, about 20 to about 90, about 20 to about 85, about 20 to about 80, about 20 to about 75, about 20 to about 70, about 20 to about 65, about 20 to about 65, about 20 to about 60, about 20 to about 55, about 20 to about 50, about 20 to about 45, about 20 to about 40, about 20 to about 35, about 20 to about 30, about 20 to about 25, about 30 to about 99, about 30 to about 90, about 30 to about 85, about 30 to about 80, about 30 to about 75, about 30 to about 70, about 30 to about 65, about 30 to about 65, about 30 to about 60, about 30 to about 55, about 30 to about 50, about 30 to about 45, about 30 to about 40, about 30 to about 35, about 40 to about 99, about 40 to about 90, about 40 to about 85, about 40 to about 80, about 40 to about 75, about 40 to about 70, about 40 to about 65, about 40 to about 65, about 40 to about 60, about 40 to about 55, about 40 to about 50, about 40 to about 45, about 50 to about 99, about 50 to about 90, about 50 to about 85, about 50 to about 80, about 50 to about 75, about 50 to about 70, about 50 to about 65, about 50 to about 65, about 50 to about 60, about 50 to about 55, about 60 to about 99, about 60 to about 90, about 60 to about 85, about 60 to about 80, about 60 to about 75, about 60 to about 70, about 60 to about 65, about 70 to about 99, about 70 to about 90, about 70 to about 85, about 70 to about 80, about 70 to about 75, about 80 to about 99, about 80 to about 90, about 80 to about 85, about 90 to about 99, or about 90 to about 95% v/v, based on the volume of the composition, of the flavoring agent. In further embodiments, the compositions contain about 10% v/v, based on the volume of the composition, of the flavoring agent.

A fragrance, i.e., compound that has an odor/scent, may optionally be included in the compositions. By doing so, the patient is obtaining nutrition using the tube, but have the psychological impression or sensation that they are ingesting a meal by tasting and smelling a specific food. The compound with an odor/scent may be selected by the patient, caregiver, or attending physician, depending on the desired smell to be elicited. For example, the patient may select the fragrance from selections available based on the particular fragrance the patient desired to taste, i.e., a menu or brochure. For example, the compound with an odor/scent may smell like a fruit, vegetable, beverage, meat, spice, extract, dairy product, starch, among others, as described above for the flavoring agent.

Water is another optional component of the compositions. In certain embodiments, the water is sterile. The term “sterile” as used herein refers to water than is free of all microorganisms (e.g., fungi, spores, or bacteria). In some embodiments, the water is distilled, deionized, prepared by reverse osmosis, or a combination thereof. The pH of sterile water is about 5 to about 7, i.e., about 5, about 5.5, about 6, about 6.5, or about 7. In some embodiments, the compositions contain about 5% v/v, based on the volume of the composition, or greater of water. In other embodiments, the compositions contain about 5, about 10, about 15, about 20, about 25, about 30, about 35, about 40, about 45, about 50, about 55, about 60, about 65, about 70, about 75, about 80, about 85, about 90, about 95, or about 99% v/v, based on the volume of the composition, of water. In further embodiments, the compositions contain about 5 to about 99, about 5 to about 90, about 5 to about 85, about 5 to about 80, about 5 to about 75, about 5 to about 70, about 5 to about 65, about 5 to about 65, about 5 to about 60, about 5 to about 55, about 5 to about 50, about 5 to about 45, about 5 to about 40, about 5 to about 35, about 5 to about 30, about 5 to about 25, about 5 to about 20, about 5 to about 15, about 5 to about 10, about 10 to about 99, about 10 to about 90, about 10 to about 85, about 10 to about 80, about 10 to about 75, about 10 to about 70, about 10 to about 65, about 10 to about 65, about 10 to about 60, about 10 to about 55, about 10 to about 50, about 10 to about 45, about 10 to about 40, about 10 to about 35, about 10 to about 30, about 10 to about 25, about 10 to about 20, about 10 to about 15, about 20 to about 99, about 20 to about 90, about 20 to about 85, about 20 to about 80, about 20 to about 75, about 20 to about 70, about 20 to about 65, about 20 to about 65, about 20 to about 60, about 20 to about 55, about 20 to about 50, about 20 to about 45, about 20 to about 40, about 20 to about 35, about 20 to about 30, about 20 to about 25, about 30 to about 99, about 30 to about 90, about 30 to about 85, about 30 to about 80, about 30 to about 75, about 30 to about 70, about 30 to about 65, about 30 to about 65, about 30 to about 60, about 30 to about 55, about 30 to about 50, about 30 to about 45, about 30 to about 40, about 30 to about 35, about 40 to about 99, about 40 to about 90, about 40 to about 85, about 40 to about 80, about 40 to about 75, about 40 to about 70, about 40 to about 65, about 40 to about 65, about 40 to about 60, about 40 to about 55, about 40 to about 50, about 40 to about 45, about 50 to about 99, about 50 to about 90, about 50 to about 85, about 50 to about 80, about 50 to about 75, about 50 to about 70, about 50 to about 65, about 50 to about 65, about 50 to about 60, about 50 to about 55, about 60 to about 99, about 60 to about 90, about 60 to about 85, about 60 to about 80, about 60 to about 75, about 60 to about 70, about 60 to about 65, about 70 to about 99, about 70 to about 90, about 70 to about 85, about 70 to about 80, about 70 to about 75, about 80 to about 99, about 80 to about 90, about 80 to about 85, about 90 to about 99, or about 90 to about 95% v/v, based on the volume of the composition, of water. In yet other embodiments, the compositions contain about 60 to about 80% v/v, based on the volume of the composition, of water. In still further embodiments, the compositions contain about 65 to about 75% v/v, based on the volume of the composition, of water. In other embodiments, the compositions contain about 70% v/v, based on the volume of the composition, of water.

The compositions described herein may further contain one or more of a preservative, surfactant, dental hygiene reagent, sweetener, solubilizing agent, buffer, antimicrobial, dye (coloring agent), moisturizer, thickening agent, among others.

In some embodiments, the compositions contain a preservative. Examples of preservatives include, without limitation, benzoic acid or salts thereof, sorbic acid or salts thereof, or parabens. In certain aspects, the preservative is a benzoic acid salt such as sodium benzoate. In other aspects, the preservative is a sorbic acid salt such as potassium sorbate. In further aspects, the preservative is a paraben such as methyl paraben, ethyl paraben, propyl paraben, or butyl paraben. In yet other aspects, the preservative is potassium sorbate. In some embodiments, the compositions contain about 0.01 to about 5% v/v, based on the volume of the composition, of the preservative. In other embodiments, the compositions contain about 0.05, about 0.1, about 0.5, about 1, about 2, about 3, about 4, or about 5% v/v, based on the volume of the composition, of the preservative. In further embodiments, the compositions contain about 0.01 to about 4, about 0.01 to about 3, about 0.01 to about 2, about 0.01 to about 2, about 0.01 to about 1, about 0.01 to about 0.5, about 0.01 to about 0.1, about 0.01 to about 0.05, about 0.05 to about 5, about 0.05 to about 4, about 0.05 to about 3, about 0.05 to about 2, about 0.05 to about 1, about 0.05 to about 0.5, about 0.05 to about 0.1, about 0.1 to about 5, about 0.1 to about 4, about 0.1 to about 3, about 0.1 to about 2, about 0.1 to about 1, about 0.1 to about 0.5, about 0.5 to about 5, about 0.5 to about 4, about 0.5 to about 3, about 0.5 to about 2, about 0.5 to about 1, about 1 to about 5, about 1 to about 4, about 1 to about 3, about 1 to about 2, about 2 to about 5, about 2 to about 4, about 2 to about 3, about 3 to about 5, about 3 to about 4, or about 4 to about 5% v/v, based on the volume of the composition, of the preservative. In still other embodiments, the composition contains about 0.1% v/v, based on the volume of the composition, of preservative.

In other embodiments, the compositions contain a surfactant. Examples of surfactants include, without limitation, sodium lauryl sulfate, sodium dodecyl sulfate, polysorbate 80, or polysorbate 20, among others. In certain aspects, the surfactant is sodium lauryl sulfate. In other aspects, the surfactant is sodium dodecyl sulfate. In further aspects, the surfactant is polysorbate 80. In yet other aspects, the surfactant is polysorbate 20. In some embodiments, the compositions contain about 0.01 to about 5% v/v, based on the volume of the composition, of the surfactant. In other embodiments, the compositions contain about 0.05, about 0.1, about 0.5, about 1, about 2, about 3, about 4, or about 5% v/v, based on the volume of the composition, of the surfactant. In further embodiments, the compositions contain about 0.01 to about 4, about 0.01 to about 3, about 0.01 to about 2, about 0.01 to about 2, about 0.01 to about 1, about 0.01 to about 0.5, about 0.01 to about 0.1, about 0.01 to about 0.05, about 0.05 to about 5, about 0.05 to about 4, about 0.05 to about 3, about 0.05 to about 2, about 0.05 to about 1, about 0.05 to about 0.5, about 0.05 to about 0.1, about 0.1 to about 5, about 0.1 to about 4, about 0.1 to about 3, about 0.1 to about 2, about 0.1 to about 1, about 0.1 to about 0.5, about 0.5 to about 5, about 0.5 to about 4, about 0.5 to about 3, about 0.5 to about 2, about 0.5 to about 1, about 1 to about 5, about 1 to about 4, about 1 to about 3, about 1 to about 2, about 2 to about 5, about 2 to about 4, about 2 to about 3, about 3 to about 5, about 3 to about 4, or about 4 to about 5% v/v, based on the volume of the composition, of the surfactant. In still other embodiments, the composition contains about 0.1% v/v, based on the volume of the composition, of surfactant.

In further embodiments, the compositions contain a dye or a coloring agent. The term “dye” as used herein refers to a reagent that imparts a color to the composition. Dyes that may be used as described herein include those approved for use in food products including those described in Summary of Color Additives for Use in the United States in Foods, Drugs, Cosmetics, and Medical Devices”, Title 21 of the Code of Federal Regulations Parts 73, 74, 81 and 82, Oct. 21, 2021. Examples of suitable dues include, without limitation, annatto extract, dehydrated beets, canthaxanthin, caramel, β-apo-8′-carotenal, β-carotene, cochineal extract, carmine, sodium copper chlorophyllin, toasted partially defatted cooked cottonseed flour, ferrous gluconate, ferrous lactate, grape color extract, grape skin extract, synthetic iron oxide, fruit juice, vegetable juice, carrot oil, paprika, paprika oleoresin, mica-based pearlescent pigments, riboflavin, saffron, spirulina extract, titanium dioxide, tomato lycopene extract, turmeric, turmeric oleoresin, FD&C Blue No. 1, FD&C Blue No. 2, FD&C Green No. 3, Orange B, Citrus Red No. 2, FD&C Red No. 3, FD&C Red No. 40, FD&C Yellow No. 5, or FD&C Yellow No. 6. In some embodiments, the compositions contain about 0.005 to about 1% v/v, based on the volume of the composition, of the coloring agent. In other embodiments, the compositions contain about 0.005, about 0.001, about 0.005, about 0.01, about 0.05, about 0.1, about 0.5, or about 1% v/v, based on the volume of the composition, of the coloring agent. In further embodiments, the compositions contain about 0.005 to about 0.5, about 0.005 to about 0.1, about 0.005 to about 0.05, about 0.005 to about 0.01, about 0.01 to about 1, about 0.01 to about 0.5, about 0.01 to about 0.1, about 0.01 to about 0.05, about 0.05 to about 1, about 0.05 to about 0.5, about 0.05 to about 0.1, about 0.1 to about 1, about 0.1 to about 0.5, or about 0.5 to about 1% v/v, based on the volume of the composition, of the coloring agent. In still other embodiments, the composition contains about 0.1% v/v, based on the volume of the composition, of the coloring agent.

In yet other embodiments, the compositions contain a dental hygiene reagent. Examples of dental hygiene reagents include, anticaries agents, desensitizing agents, abrasive, detergents, thickening agent, or peroxide. In certain aspects, the dental hygiene reagent is an anticaries agents such as a fluoride-containing compound (e.g., sodium monofluorophosphate, sodium fluoride or stannous fluoride). In other aspects, the dental hygiene reagent is a desensitizing agent such as stannous fluoride, calcium sodium phosphosilicate, hydroxyapatite, arginine, strontium, or potassium. In further aspects, the dental hygiene reagent is an abrasive such as calcium carbonate, dehydrated silica gel, hydrated aluminum oxide, magnesium carbonate, phosphate salt or silicate. In still other aspects, the dental hygiene reagent is a detergent such as sodium lauryl sulfate or sodium N-lauryl sarcosinate. In still further aspects, the dental hygiene reagent is a humectant such as glycerol, propylene glycol or sorbitol). In other aspects, the dental hygiene agent is a thickening agent such as a mineral colloid, seaweed colloid, natural gum, or synthetic cellulose. In further aspects, the dental hygiene reagent includes a peroxide such as hydrogen peroxide and carbamide peroxide.

In still further embodiments, the compositions contain a sweetener. Examples of sweeteners include, without limitation, sugars (e.g., sucrose, fructose, glucose, maltose, lactose) or sugar alcohols/polyols (e.g., maltitol, lactitol, sorbitol), in particular sorbitol.

In other embodiments, the compositions contain a solubilizing agent.

In other embodiments, the compositions contain a buffer or pH adjustor. In some embodiments, the buffer is sodium citrate. The compositions may contain about 0.01 to about 1% v/v, based on the volume of the composition, of the buffer. In some embodiments, the compositions contain about 0.01, about 0.05, about 0.1, about 0.2, about 0.3, about 0.4, about 0.5, about 0.6, about 0.7, about 0.8, about 0.9, or about 1% v/v, based on the volume of the composition, of the buffer. In other embodiments, the compositions contain about 0.01 to about 0.75, about 0.01 to about 0.5, about 0.01 to about 0.25, about 0.01 to about 0.1, about 0.01 to about 0.05, about 0.05 to about 1, about 0.05 to about 0.75, about 0.05 to about 0.5, about 0.05 to about 0.25, about 0.05 to about 0.1, about 0.1 to about 1, about 0.1 to about 0.75, about 0.1 to about 0.5, about 0.1 to about 0.25, about 0.25 to about 1, about 0.25 to about 0.75, about 0.25 to about 0.5, about 0.5 to about 1, about 0.5 to about 0.75, or about 0.75 to about 1% v/v, based on the volume of the composition, of the buffer. In further embodiments, the compositions contain about 0.25% v/v, based on the volume of the composition, of the buffer.

In further embodiments, the compositions contain an antimicrobial. Examples of antimicrobials include, without limitation, triclosan, cetylpyridinium chloride or alcohol.

In yet other embodiments, the compositions contain a moisturizer that moistens the mouth. Examples of moisturizers include, without limitation, citric acid.

The compositions may also contain one or more vitamins or minerals that are ingestible by a human. Examples of vitamins include, without limitation, cholecalciferol (vitamin D3), zinc (e.g., zinc gluconate hydrate), or combinations thereof. In some embodiments, the compositions contain about 0.0001 to about 20% v/v, based on the volume of the composition, of the one or more vitamin. In other embodiments, the compositions contain about 0.0001, about 0.0005, about 0.001, about 0.002, about 0.005, about 0.001, about 0.05, about 0.01, about 0.5, about 1, about 5, about 10, about 15, or about 20% v/v, based on the volume of the composition, of the one or more vitamin. In further embodiments, the compositions contain about 0.001 to about 20, about 0.001 to about 15, about 0.001 to about 10, about 0.001 to about 5, about 0.001 to about 1, about 0.001 to about 0.5, about 0.001 to about 0.1, 0.001 to about 0.05, about 0.001 to about 0.01, about 0.001 to about 0.005, about 0.005 to about 20, about 0.005 to about 15, about 0.005 to about 10, about 0.005 to about 5, about 0.005 to about 1, about 0.005 to about 0.5, about 0.005 to about 0.1, about 0.005 to about 0.05, about 0.005 to about 0.01, about 0.01 to about 20, about 0.01 to about 15, about 0.01 to about 10, about 0.01 to about 5, about 0.01 to about 1, about 0.01 to about 0.5, about 0.01 to about 0.1, about 0.01 to about 0.05, about 0.05 to about 20, about 0.05 to about 15, about 0.05 to about 10, about 0.05 to about 5, about 0.05 to about 1, about 0.05 to about 0.5, about 0.05 to about 0.1, about 0.1 to about 20, about 0.1 to about 15, about 0.1 to about 10, about 0.1 to about 5, about 0.1 to about 5, about 0.1 to about 1, about 0.1 to about 0.5, about 0.5 to about 20, about 0.5 to about 15, about 0.5 to about 10, about 0.5 to about 5, about 0.5 to about 1, about 1 to about 20, about 1 to about 15, about 1 to about 10, about 1 to about 5, about 5 to about 20, about 5 to about 15, about 5 to about 10, about 10 to about 20, about 10 to about 15, or about 15 to about 20% v/v, based on the volume of the composition, of the one or more vitamin. In yet other embodiments, the compositions contain about 10% v/v, based on the volume of the composition, of the one or more vitamin. In still further embodiments, the compositions contain about 10% v/v, based on the volume of the composition, of zinc gluconate hydrate and about 0.002% v/v, based on the volume of the composition, of vitamin D.

“The compositions may also contain a stabilizing agent. Examples of stabilizing agents include, without limitation, xanthan gum. The compositions may contain about 0.01 to about 1% v/v, based on the volume of the composition, of the stabilizing agent. In some embodiments, the compositions contain about 0.01, about 0.05, about 0.1, about 0.2, about 0.25, about 0.3, about 0.4, about 0.5, about 0.6, about 0.7, about 0.75, about 0.8, about 0.9, or about 1% v/v, based on the volume of the composition, of the stabilizing agent. In other embodiments, the compositions contain about 0.01 to about 0.75, about 0.01 to about 0.5, about 0.01 to about 0.25, about 0.01 to about 0.1, about 0.01 to about 0.05, about 0.05 to about 1, about 0.05 to about 0.75, about 0.05 to about 0.5, about 0.05 to about 0.25, about 0.05 to about 0.1, about 0.1 to about 1, about 0.1 to about 0.75, about 0.1 to about 0.5, about 0.1 to about 0.25, about 0.25 to about 1, about 0.25 to about 0.75, about 0.25 to about 0.5, about 0.5 to about 1, about 0.5 to about 0.75, or about 0.75 to about 1% v/v, based on the volume of the composition, of stabilizing agent. In further embodiments, the compositions contain about 0.25% v/v, based on the volume of the composition, of the stabilizing agent.

In one embodiments, the composition contains water, a flavoring agent, one or more vitamin, propylene glycol, one or more preservative, one or more surfactant, one or more, one or stabilizing agent, and one or more coloring agent.

In another embodiment, the composition contains water, one or more flavoring agent, zinc gluconate hydrate, propylene glycol, polysorbate 80, xanthan gum, sodium citrate, potassium sorbate, coloring agent, and vitamin D.

In a further embodiment, the composition contains:

(i) about 5% v/v or greater, based on the volume of the composition (preferably about 5 about 50% v/v, or preferably about 5 to about 25% v/v, or preferably about 5 to about 10% v/v), of propylene glycol;

(ii) at least about 5% v/v (preferably about 5 to about 20% v/v), based on the volume of the composition, of the flavoring agent;

(iii) about 5% v/v (preferably about 50 to about 90, or preferably about 60 to about 80% v/v), based on the volume of the composition, or greater of water;

(iv) about 0.01 to about 5% v/v (preferably about 0.01 to about 1% v/v), based on the volume of the composition, of the preservative, about 0.005 to about 1% v/v (preferably about 0.1 to about 1% v/v), based on the volume of the composition, of the coloring agent, (v) about 0.01 to about 1% v/v (preferably about 0.1 to about 1% v/v), based on the volume of the composition, of the buffer;

(vi) about 0.0001 to about 20% v/v (preferably about 0.001 to about 20% v/v), based on the volume of the composition, of the one or more vitamin;

(vii) about 0.01 to about 1% v/v (preferably about 0.05 to about 1% v/v), based on the volume of the composition, of the stabilizing agent; and (viii) about 0.1 to about 5% v/v (preferably 0.1 to about 2% v/v), based on the volume of the composition, of the surfactant.

In still another embodiment, the composition contains about 70% v/v, based on the volume of the composition, of water, 10% v/v, based on the volume of the composition, of a flavoring agent, 10% v/v, based on the volume of the composition, of zinc gluconate hydrate, 7.5% v/v, based on the volume of the composition, of propylene glycol, 1% v/v, based on the volume of the composition, of polysorbate 80, 0.25% v/v, based on the volume of the composition, xanthan gum, 0.25% v/v, based on the volume of the composition, of sodium citrate, 0.1% v/v, based on the volume of the composition, of potassium sorbate, 0.1% v/v, based on the volume of the composition, of a coloring agent, and 0.002% v/v, based on the volume of the composition, of vitamin D.

The compositions described herein desirably are sterile.

### Devices

Although the compositions described herein may be supplied as a neat composition, the present disclosure also contemplates devices comprising the composition described herein. In some embodiments, the device is a swab or film, e.g., a consumable film. The term “consumable film” as used herein refers to physiologically acceptable film through contact with the oral cavity and the natural human salivation and moisturizing response in the mouth of a patient. In some embodiments, the consumable film is a film described in U.S. Pat. No. 6,596,298, which is hereby incorporated by reference.

In other embodiments, the device is a swab. In further embodiments, the device may be selected from the swabs described in, e.g., United Kingdom Patent No. 4795421, U.S. Pat. No. D-468424, US Patent Publication No. 20160038348, U.S. Pat. No. 4,795,421, “Specimen Collection Swab|Nasopharyngeal,” Datasheet, Typenex Medical, 2021; “Specimen Collection Swab|Oropharyngeal,” Datasheet, Typenex Medical, 2021; “Specimen Collection Swab|Nasal,” Datasheet, Typenex Medical, 2021; “Innovation in Packaging,” Catalog 2.2, DaklaPack Europe; “Supplies of FDA-Approved Swabs and Transport Medica,” Thomasnet.com, 2021; “Puritan Medical Catalog,” puritanmedproducts.com, March 2019; “Puritan Diagnostics Catalog,” puritanmedproducts.com, Puritan Medical Products Co. LLC, March 2019; “Puritan Forensics Catalog,” puritanmedproducts.com, Puritan Medical Products Co. LLC, March 2019; “Puritan Environmental Catalog,” puritanmedproducts.com, Puritan Medical Products Co. LLC, March 2019; and “Disposable Medical Products Catalog,” Dynarex, 2019, which are incorporated by reference herein. In yet other embodiments, the swab is a portable hand-held applicator (e.g., wooden or plastic stick), having a sponge, foam or pad attached thereto that contains the composition/flavoring agent.

The particular swab or consumable film for use herein may be selected by one skilled in the art. Desirably, the swab or consumable film is capable of absorbing the composition described herein. In some embodiments, the swab or consumable film absorbs sufficient amounts of the compositions so that it is saturated, i.e., incapable of absorbing any more composition. In other embodiments, the swab or consumable film absorbs the composition until it is about 80% v/v filled. In further embodiments, the swab or consumable film absorbs the composition until it is at least about 80, about 85, about 90, about 95, or about or 99% v/v filled. In yet other embodiments, the swab or consumable film absorbs the composition until it is at least about 80 to about 100, about 80 to about 99, about 80 to about 95, about 80 to about 90, about 80 to about 85, about 85 to about 100, about 85 to about 99, about 85 to about 95, about 85 to about 90, about 90 to about 100, about 90 to about 99, about 90 to about 95, about 95 to about 100, about 95 to about 99, or about 99 to about 100% v/v filled. The swab or consumable film made be made of any material that is capable of absorbing the composition. In certain aspects, the swab comprises a natural or synthetic material. In further aspects, the swab is a synthetic material such as a composite or foam. In yet other aspects, the swab is a natural materials such as cotton. In other aspects, the swab comprises foam.

The patient, caregiver, or attending physician may apply the device to the oral mucous membrane of the patient. In some aspects, the patient is capable of applying the device to the oral mucous membrane. In other aspects, a caregiver applies the device to the oral mucous membrane of the patient. In further aspects, an attending physician applies the device to the oral mucous membrane of the patient.

The device may be applied using skill in the art. In certain embodiments, a swab or consumable film containing the composition is placed in the mouth of the patient and the liquid contained therein sucked out. In further embodiments, a film containing the composition is placed in the patient's mouth and the film dissolved using the patient's saliva. In still other embodiments, a drop of the composition is placed in the patient's mouth. A device can be pre-loaded (e.g., impregnated) with the composition such that a device can be removed from its packaging and immediately used with a patient. Alternatively, a device can be packaged “dry” (i.e., free of the composition) and can be contacted with the composition at the time and/or location of use. A device can also include composition pre-stored therein, e.g., within an internal chamber, which stored composition can be liberated (e.g., by breaking a membrane or other storage chamber) at the time and/or location of use.

As for the compositions, the device described herein may be sterile.

### Methods

The compositions and devices described herein are useful in methods for moistening oral mucous membranes of patients in need thereof. In doing so, the compositions and devices lubricate the mouth of the patient, thereby providing relief from dryness that typically accompanies patients on feeding tubes.

The patients may be unable to swallow or begin to choke after small amounts of water or saliva enter the esophagus. In some embodiments, the patients have a feeding tube. One of skill in the art would understand the types of feeding tubes utilized by patients who are unable to swallow food or liquids. Examples of tubes include, without limitation, nasogastric (NG) tubes, nasoduodenal tubes, nasojejunal (NJ) tubes, gastrostomy tubes, gastrojejunal (G-J) tubes, or jejunal tubes. In certain aspects, the tube is a nasogastric tube. In other aspects, the tube is a nasoduodenal tube. In further aspects, the tube is a nasojejunal tube. In yet other aspects, the tube is a gastrostomy tube. In still further aspects, the tube is a gastrojejunal tube. In other aspects, the tube is a jejunal tube. In further aspects, the tube is a total parenteral nutrition (TPN) tube, such as a TPN intravenous tube. The feeding tube may administer the food or liquids by gravity, intravenously, via a bolus, or pump.

In contrast to other compositions and devices available in the art, the instant compositions and devices stimulate the appetite of the patient and/or elicit the patient's sense of taste and smell. Desirably, the compositions and devices also moisture without creating an abundance of saliva which could result in choking. In certain aspects, the compositions and devices stimulate the appetite of a patient in need thereof. In other aspects, the compositions and devices stimulate and/or elicit a sense of taste, smell, or combinations thereof in a patient in need thereof.

Although the compositions and methods provide much needed physical aids to patients, doing so also affects the patients psychologically. As discussed herein, patients requiring feeding tubes are at a higher risk for sadness or depression. Often, the sadness or depression is linked with the inability to physically consume food or to enjoy the tastes and smells associated with food. Thus, the disclosure provides methods for improving a disposition of a patient unable to taste, smell, chew and/or swallow food or excess liquid. In some embodiments, the patient is unable to taste. In other embodiments, the patient is unable to chew. In further embodiments, the patient is unable to swallow food. In yet other embodiments, the patient is unable to swallow excess liquid. In still further embodiments, the patient is unable to smell their food through their tube feedings. The term “excess liquid” as used herein refers to an amount of liquid that induces a choking response when swallowing. The amount of excess liquid depends on the patient, including the patient's condition. These methods comprise applying the compositions or devices described herein to the oral mucous membrane of the patient. In certain aspects, the patient's disposition is improved after only one application of the composition or device. However, the compositions or devices may be used by the patient as often as needed. In other aspects, the compositions of devices are used hourly, daily, weekly, monthly, yearly, or combinations thereof. The patient, caregiver, or attending physician would be able to determine how often the patient should use the compositions or devices/

The methods include applying the compositions or device described herein to the oral mucous membrane of the patient. One of skill in the art would readily understand how to apply the compositions of devices to the patient's oral mucous membrane. In some embodiments, a caregiver or attending physical applies the composition or device to the mucous membrane. In other embodiments, the patient applies the composition or device to the mucous membrane.

### Aspects

Aspect 1. A composition for moistening an oral mucous membrane of a patient in need thereof, comprising propylene glycol and a flavoring agent.

Aspect 2. The composition of Aspect 1, that lacks glycerin.

Aspect 3. The composition of Aspect 1 or 2, wherein the flavoring agent has an odor.

Aspect 4. The composition of any one of the preceding Aspects, wherein the composition further comprises a compound that has an odor.

Aspect 5. The composition of any one of the preceding Aspects, wherein the composition further comprises water.

Aspect 6. The composition of any one of the preceding Aspects, wherein the composition further comprises one or more of a preservative, surfactant, dental hygiene reagent, sweetener, solubilizing agent, buffer, antimicrobial, dye, and salivation stimulator.

Aspect 7. The composition of any one of the preceding Aspects, that is sterile.

Aspect 8. A device comprising the composition of any one of Aspects 1-7.

Aspect 9. The device of Aspect 8, that is a swab or film, preferably a swab.

Aspect 10. The device of Aspect 9, wherein the swab comprises foam or cotton, preferably foam.

Aspect 11. The device of any one of Aspects 8-10, that is sterile.

Aspect 12. A method for moistening an oral mucous membrane of a patient in need thereof, comprising applying the composition of any one of Aspects 1-7 or the device of any one of Aspects 8-11 to the oral mucous membrane of the patient.

Aspect 13. A method for stimulating the appetite of a patient in need thereof, comprising applying the composition of any one of Aspects 1-7 or the device of any one of Aspects 8-11 to the oral mucous membrane of the patient.

Aspect 14. A method for improving a disposition of a patient unable to taste, smell, chew and/or swallow food or excess liquid, comprising applying the composition of any one of Aspects 1-7 or the device of any one of Aspects 8-11 to the oral mucous membrane of the patient.

Aspect 15. The method of any one of Aspects 12-14, wherein the patient is a child.

Aspect 16. The method of any one of Aspects 12-14, wherein the patient is an adult.

Aspect 17. The method of Aspect 16, wherein the patient is a geriatric adult.

Aspect 18. The method of any one of Aspects 12-17, wherein the patient is unable to swallow.

Aspect 19. The method of any one of Aspects 12-18, wherein the patient has a feeding tube.

Aspect 20. The method of Aspect 19, wherein the tube is a nasogastric tube, nasoduodenal tube, nasojejunal tube, gastrostomy tube, gastrojejunal tube, or jejunal tube.

### EXAMPLES

### Example 1

A composition is prepared containing 50% v/v of propylene glycol and 50% v/v of a coffee flavoring agent. The composition is mixed and applied to a swab or consumable film in a sterile environment, packaged and sealed.

### Example 2

A foam swab or consumable film is dipped into the composition of Example 1 until it is about 80% v/v filled. The swab or consumable film is not dripping with the composition, i.e., saturated.

### Example 3

A caregiver or patient applies the foam swab or consumable film of Example 2 to the oral mucous membrane of a patient on a feeding tube.

### Example 4

A caregiver or patient swabs their mouth with the foam swab or consumable film of Example 2 prior to or at the onset of their g-tube feeding.

### Example 5

Five types of swabs (Samples A-F) containing one or more of the following ingredients were prepared:
Distilled water
Zinc gluconate hydrate
Natural flavorings (vegan, gluten and nut/soy free)
Vitamin D3
Sodium citrate
Propylene glycol
Polysorbate 80
Xanthan gum
Potassium sorbate

The swabs were basic and single flavored. Sample A was egg flavored, sample B was bacon flavored, sample C was espresso coffee flavored, sample D was cooked chicken flavored, sample E was chardonnay wine flavored, and sample F was chocolate flavored.

Each swab was labeled with the Sample letter. Patients were instructed to open the plastic bag, remove the swab, and lightly swab the inside of the mouth (oral cavity) while receiving meal nutrition.

For Samples A, B or C use the swab during the morning meal.

For Sample D, use the swab either during the lunchtime or evening meal.

For Sample E, (only for adults 21 or older) use the swab during the lunchtime or evening meal.

For Sample F, use the swab toward the END of the lunchtime or evening meal or during the snack meal in between lunchtime and evening meals.

Each patient received either samples A, B or C in addition to F and either D or E.

The caregiver or patient evaluated the efficacy of the swab after swabbing his/her mouth by answering the following questions:

| | | | |
|---|---|---|---|
| | Somewhat or | Very or | |
| Not at all | Sometimes | Most Often | N/A |

My mouth was dry.
I enjoyed my food more.
I felt more satisfied while
eating.
My mouth felt more
moistened.
I felt more satiated (full)
after eating.
I looked more forward
to meals.
I was unable to taste
my food.
Eating was a pleasurable
experience.
I felt as though I could
taste my food.
I did not look forward
to meals.

The caregiver or patient also evaluated the effect on his/her disposition and eating response by answering the following questions:

| | | | |
|---|---|---|---|
| | Somewhat or | Very or | |
| Not at all | Sometimes | Most Often | N/A |

The swab made me feel
better physically
while eating.
The swab made me feel
better emotionally
while eating.
The swab made eating
difficult.
The swab increased my
salivation response
too much.
The swab had a positive
overall effect on my
eating experience.
The swab had a negative
overall effect on my
eating experience.

In summary, 125 surveys were sent out and 111 were returned. Prior to using the swabs:
i) 98% of patients indicated they did not enjoy their food at all.
ii) 100% of patients stated their mouth was often dry.
iii) 68% of patients did not look forward to meals at all.
iv) 100% of patients were unable to taste their food.
v) 98% of patients felt that eating was never a pleasurable experience.

Overall, patient response was overwhelmingly positive. After using the swabs, 84% of patients indicated that the swabs made them feel better physically while eating. 91% of patients indicated the swabs made them feel better emotionally while eating. 96.4% of patients indicated that the swabs had a positive overall effect on their eating experience. Patients also noted the moisturizing effect. 96.4% of patients indicated that their mouth felt more moistened.

### Example 6

A composition was prepared containing 70.8% water (distilled or reversed osmosis), 10% flavoring agent, 10% zinc gluconate hydrate, 7.5% propylene glycol, 1% polysorbate 80, 0.25% xanthan gum, 0.25% sodium citrate, 0.1% potassium sorbate, 0.1% coloring ingredient, and 0.002% cholecalciferol (vitamin D3). The composition was prepared by mix the propylene glycol and xanthan gum at high shear for 5 minutes at 25° C. Water was then added and the resultant solution was mixed for 30 minutes at high shear at 25° C. The flavoring agent, zinc gluconate hydrate, polysorbate 80, potassium sorbate, coloring ingredient, and vitamin D3 were then added to the mixed water solution and the resultant solution mixed at medium shear for 5 minutes at 25° C. The pH was then adjusted to 6-6.5 with sodium citrate.

The composition was then applied to a swab by coating the swab with the composition. Coating was performed by dipping the swab in a sample of the flavor. Once coated, swabs were allowed to dry.

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present disclosure contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes.

What is claimed is:

1. A composition for moistening an oral mucous membrane of a patient in need thereof, comprising propylene glycol and a flavoring agent.

2. The composition of claim 1, that lacks glycerin.

3. The composition of claim 1, wherein the flavoring agent has an odor.

4. The composition of claim 1, wherein the composition further comprises a compound that has an odor.

5. The composition of claim 1, wherein the composition further comprises water.

6. The composition of claim 1, wherein the composition further comprises one or more of a preservative, surfactant, dental hygiene reagent, sweetener, solubilizing agent, buffer, antimicrobial, dye, and moisturizer.

7. The composition of claim 1, that is sterile.

8. A device comprising the composition of claim 1.

9. The device of claim 8, that is a swab or film, preferably a swab or consumable film.

10. The device of claim 9, wherein the swab comprises foam or cotton.

11. The device of claim 8, that is sterile.

12. A method for moistening an oral mucous membrane of a patient in need thereof, comprising applying the composition of claim 1 to the oral mucous membrane of the patient.

13. A method for stimulating the appetite of a patient in need thereof, comprising applying the composition of claim 1 to the oral mucous membrane of the patient.

14. A method for improving a disposition of a patient unable to taste, smell, chew and/or swallow food or excess liquid, comprising applying the composition of claim 1 to the oral mucous membrane of the patient.

15. The method of claim 12, wherein the patient is a child.

16. The method of claim 12, wherein the patient is an adult.

17. The method of claim 16, wherein the patient is a geriatric adult.

18. The method of claim 12, wherein the patient is unable to swallow.

19. The method of claim 12, wherein the patient has a feeding tube.

20. The method of claim 19, wherein the tube is a nasogastric tube, nasoduodenal tube, nasojejunal tube, gastrostomy tube, gastrojejunal tube, or jejunal tube.
